(19) United States
(12) Patent Application Publication
(10) Pub. No.: US 2017/0095776 A1
(43) Pub. Date: Apr. 6, 2017

(54) FILTER MODULE OF AN EXTRACORPOREAL BLOOD TREATMENT MACHINE

(71) Applicant: B. BRAUN AVITUM AG, MELSUNGEN (DE)

(72) Inventor: MANUEL GOLDSTEIN, DRESDEN (DE)

(21) Appl. No.: 15/274,444

(22) Filed: Sep. 23, 2016

(30) Foreign Application Priority Data
Oct. 2, 2015 (DE) 10 2015 116 787.5

(51) Int. Cl. B01D 69/08; B01D 61/24; A61M 1/16; B01D 63/02

(52) U.S. Cl. CPC B01D 69/081; B01D 63/02; B01D 61/243; A61M 1/1621

(57) ABSTRACT

A filter module of an extracorporeal blood treatment machine with a filter casing in which a hollow fiber bundle is attached whose hollow fibers comprise a hollow fibre wall made of a semi-permeable membrane, and whereby in their longitudinal extension the hollow fibers comprise a number of changes in their diameter. An amplitude of 0.1 to 1 mm is achieved for the exterior diameter of the respective hollow fiber starting from the hollow fiber's axis, whereby the periodicity provided in the axial direction of the hollow fiber is within a range of 1 to 10 cm and on the inside of the filter casing at least one radial constriction is configured or provided by which the fiber bundle is locally constricted.

### CROSS REFERENCE TO RELATED APPLICATIONS

This application claims priority to German application DE 10 2015 116 787.5 filed Oct. 2, 2015, the contents of such application being incorporated by reference herein.

### FIELD OF THE INVENTION

The present invention concerns a filter module of an extracorporeal blood treatment machine, preferably a dialysis machine, with a filter casing in which a hollow fiber bundle is attached whose hollow fibers comprise a hollow fiber wall made of a semi-permeable membrane, whereby in their longitudinal extension the hollow fibers exhibit a number of changes, at least in their exterior diameter.

Background to the Invention

A key criterion of a filter module (hereinafter referred to as a dialyzer) in particular for use in a blood purification machine such as a dialysis machine is its filtering capacity. As such, it is the aim of development to increase this filtering capacity, though without lessening the operational reliability of the dialyzer or impairing its manageability. One way of increasing the filtering capacity concerns the quality of the filter membrane itself, including its positioning and the distribution of pores of varying diameters. Another possibility lies in improving/optimizing the hollow fiber geometry, especially in the longitudinal direction of the fibers.

Description of the Related Art

It is known from the state of the art that hollow fiber bundles can be constricted inside a dialyzer in parts of the overall cross-section of the bundles so as to create a kind of flow obstruction which has a regulating effect on a purification fluid such as dialysis fluid in this way. The aim is for a rinsing or purification fluid to flow across the entire filter cross-section and not just to use flow paths in parts with randomly reduced flow resistance. Furthermore it is essentially known that individual hollow fibers, as shown by way of an example in FIG. 3, can be configured or arranged within such a fiber bundle in a wave shape (undulation). The aim of this known measure is to achieve better bathing of the hollow fibers on the filtrate side (downstream) by means of suitable fiber undulation. What is more, the fiber bundle will generally comprise spacer threads which also promote bathing of the fibers.

These generally known measures have disadvantages, however.

The fiber undulation, generally two-dimensional, has the problem of adjacent fibers in the same phase. This means that rinsing fluid no longer bathes the contact surfaces, or does so insufficiently. This would cancel out the desired effect. Prevention of these laminates (i.e. the two-dimensional clinging/sticking together of hollow fiber membranes) on the feed (upstream) side in particular is therefore not possible using known undulation, or it is only possible to a limited extent.

Investigations into this problem on the part of the present applicant have led to the following insights:

Trans-membrane material transport in a hollow fiber membrane filter module of the relevant type (for example for the purpose of haemodialysis) through which fluid passes axially and which is preferably operated according to the counter-current principle is impaired among other things due to the fact that
membrane areas on the filtrate side are not subject to maximum circulation by a rinsing solution (e.g. dialysis fluid) in spite of fiber undulation because fibers of the fiber bundle are locally aligned in parallel and in the same phase over certain longitudinal fiber extensions, so that wave crests or wave troughs come to lie directly inside and alongside one another and
the flow on the feed side, in spite of the fiber undulation described, is in the laminar flow area, as a result of which the substances to be removed (e.g. molecules subject to urinary excretion in the case of a dialysis) cannot reach the fiber wall, because if there is an insufficient propelling force (e.g. a concentration gradient), the various laminates cannot be permeated.

### SUMMARY OF THE INVENTION

Based on this problem, an object of the present invention is to change the geometry of a hollow fiber membrane in such a way that it is possible to achieve an increase in the filter capacity of a filter module (dialyzer) fitted with it.

This object is achieved by means of a filter module with the characteristics of the independent claim. Advantageous embodiments of the invention are the subject of the dependent claims.

The underlying notion of the present invention to achieve the object at hand is thus to ensure that
in particular/at least on the filtrate side (downstream) there are only individual/minimum points of contact between the hollow fibers so as to promote circulation flow of the rinsing fluid (i.e. a reduction of the so-called dead space (area not bathed) automatically improves the purification capacity/filter, capacity of the filter module) and
in particular/at least on the feed side (upstream) there is a (pre-) defined/reasonable turbulence/mixing within the flow of fluid intended for purification, such as blood, in the hollow fibers, so as to increase the probability that substances to be removed are transported in an outward radial direction, i.e. towards the fiber wall of the hollow fiber (a shortened diffusion path achieves improved rinsing capacity of the filter/filter module).

In the present case, the above-formulated underlying notion of the present invention is implemented in constructional terms in that the hollow fiber is subjected to a number of changes/variations (wider—narrower) in its longitudinal extension, at least in terms of its exterior diameter. The effect of this is that two neighbouring hollow fibers, even in a parallel position, do not lie against each other flatly (in linear fashion) but essentially at certain points.

Preferably, the changes in the longitudinal direction of the fibers occur periodically, whereby radial extensions and radial constrictions are produced in alternation along the individual hollow fiber, which may be superimposed by external constrictions on a fiber bundle, formed from a large number of hollow fibers according to aspects of the invention.

Further preferably, the thickness of the hollow fiber wall remains (essentially) constant in its longitudinal extension. This means that the constructional design of the hollow fiber exterior diameter is mirrored in the hollow fiber interior diameter.

In other words, the hollow fiber membrane acquires a number of bulges and constrictions along its longitudinal extension, while the hollow fiber wall thickness remains (essentially) constant. The effect of this is that within the hollow fiber in its longitudinal extension, a number of interior cross-section reductions and subsequent interior cross-section expansions are formed, giving rise to a nozzle effect in the transition area. A fluid (e.g. blood) flowing through such a hollow fiber will therefore undergo an increase in flow speed in the area of each of these interior cross-section reductions, followed by expansion in the turbulent flow area. In this way, substances for removal which are contained in the fluid flowing through the hollow fiber can be more effectively guided to the interior fiber wall.

### BRIEF DESCRIPTION OF THE DRAWINGS

The invention is best understood from the following detailed description when read in connection with the accompanying drawings. Included in the drawings are the following figures:

FIG. 1 shows a filter module, preferably a dialyzer, according to aspects of the invention, which in the present case is adapted for use in a blood treatment machine, preferably a dialysis machine,

FIG. 2 shows a longitudinal section of a hollow fiber according to a preferred embodiment of the present invention and

FIG. 3 shows a longitudinal section of a hollow fiber according to the state of the art.

### DETAILED DESCRIPTION OF THE PREFERRED EMBODIMENTS

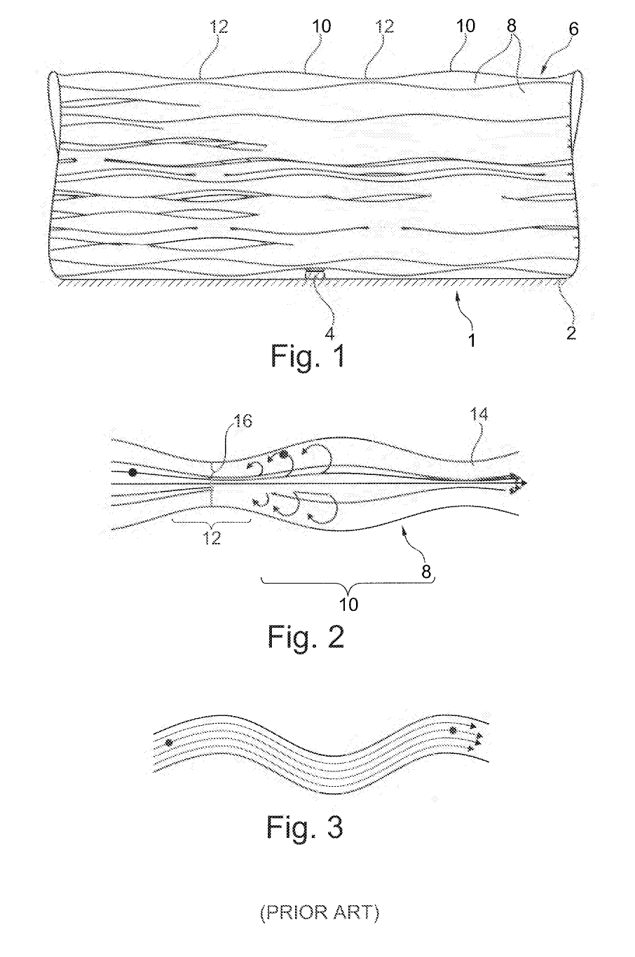

FIG. 1 is shows a filter module 1 in schematic form. The latter therefore has a preferably cylindrical filter casing 2 whose axial ends are closed with a casing cover (not shown in more detail here because it is sufficiently familiar from the state of the art). Along the filter casing 2 on the inside there are a number of axially spaced radial protrusions 4 (only one protrusion is shown as an example), by which the interior cross-section of the filter casing 2 is locally diminished.

A bundle 6 of hollow fibers 8 is inserted in the filter casing 2, whereby a total flow lumen is formed inside the hollow fibers 8 and a total flow lumen is formed outside the hollow fibers 8 inside the filter casing 2. The two total flow lumens can be connected separately from one another via input and outlet connections (not shown in any further detail because they are sufficiently familiar from the state of the art) in the filter casing 2 and/or in the casing covers to a blood treatment machine such as a dialysis machine and to the blood circulation of a patient, in such a way that, according to the counter-current principle, blood can be directed through the hollow fibers 8 and rinsing fluid can be directed through the filter casing 2 outside the hollow fibers 8.

The radially inwardly projecting protrusions 4 have the effect of causing a local constriction of the hollow fiber bundle 6 in each case, whereby in these constricted areas the overall flow cross-section outside the hollow fibers 8 is diminished, forming a kind of flow obstacle with a higher flow resistance. This serves to ensure that a rinsing fluid on the axial inlet side of the filter module 1 evenly fills the entire flow lumen outside the hollow fibers 8.

As can be further seen from FIG. 1, the hollow fibers 8 are not configured with a constant exterior cross-section along their length but exhibit at least at the hollow fiber exterior diameter (periodically) alternating radial expansions/bulges 10 and narrowings/constrictions 12, which preferably exhibit a continuous transition between each other without forming steps or edges. The hollow fibers 8 with such an exterior diameter progression are preferably positioned inside the hollow fiber bundle 6 in such a way that the bulges 10 and constrictions>12 of different hollow fibers 8 become chaotically arranged such that neighbouring hollow fibers 8 essentially only come into contact with each other at certain points along their longitudinal extension.

In reference to FIG. 2, each such hollow fiber 8 has a semi-permeable membrane wall 14 which exhibits an (essentially) constant wall thickness along its longitudinal extension so that the radial expansions 10 and constrictions 12 of the hollow fiber exterior diameter are reflected in the hollow fiber interior diameter. In this way, nozzle-like narrowings 16 are formed on the inside of the hollow fiber, by means of which the fluid flowing through the hollow fiber 8 moves at a higher speed than it does in the area of the radial bulges 10. This in turn causes a turbulence of the fluid flow on the downstream side of the radial narrowings 12, naturally assuming sufficient knowledge of the, flow conditions and the viscosity of the fluid.

Due to the alternately positioned bulges 10 and narrowings 12, neighbouring and adjacent hollow fibers 8 essentially remain at a distance and can therefore be subjected to sufficient circulation with rinsing fluid (dialysis fluid). Furthermore, this particular construction causes a flow within the hollow fiber 8 locally in the turbulent area which causes substances intended for removal to reach the semi-permeable membrane wall 14 of the hollow fiber 8 more effectively. Both effects contribute to increasing filter capacity without enlarging the filter module 1 overall, which would make it less manageable.

It is preferable for the exterior diameter of the hollow fiber 8 to have an amplitude of 0.1 to 1 mm. An advantageous periodicity in the axial direction of the hollow fiber 8 is a range of 0.1 to 10 cm.

In summary, a hollow fiber is disclosed with a hollow fiber wall made of a semi-permeable membrane, preferably for use in a filter module of a blood purification machine, whereby the hollow fiber exhibits in its longitudinal extension a number of changes at least in its exterior diameter.

1-5. (canceled)

6. Filter module of an extracorporeal blood treatment machine, the filter module comprising:
a filter casing;
a hollow fiber bundle within the filter casing, wherein each fiber of a plurality of fibers within the hollow fiber bundle comprises a fiber wall made of a semi-permeable membrane and exhibits a number of exterior diameter changes having a periodicity between 1 and 10 cm in a longitudinal direction of the hollow fiber, the exterior diameter changes having an amplitude of 0.1 to 1 mm from a longitudinal axis of the hollow fiber; and
at least one radial constriction on the inside of the filter casing that locally constricts the hollow fiber bundle.

7. The filter module according to claim 6, wherein the exterior diameter changes occur periodically in the longitudinal direction of each hollow fiber, thereby giving rise to alternating radial expansions and radial constrictions along the hollow fiber.

8. The filter module according to claim 6, wherein the thickness of the hollow fiber wall in the longitudinal direction is essentially constant, so that the interior diameter of the hollow fiber changes subject to the exterior diameter changes of the hollow fiber.

9. The filter module according to claim 7, wherein the exterior diameter changes in the longitudinal direction of the hollow fiber are applied on a continuous basis.

10. The filter module according to claim 9, wherein the exterior diameter changes in the longitudinal direction are rounded.

11. The filter module according to claim 7, wherein the transition from constrictions to subsequent expansions is configured in such a way that a fluid flow inside the hollow fiber is swirled at a known fluid viscosity and at a predefined flow speed.

12. The filter module according to claim 6, wherein the extracorporeal blood treatment machine is a dialysis machine.
